(19) United States
(12) Patent Application Publication
(10) Pub. No.: US 2012/0041402 A1
(43) Pub. Date: Feb. 16, 2012

(54) DRAPEABLE WOUND DRESSING

(75) Inventor: Bryan Greener, York (GB)

(73) Assignee: Smith & Nephew PLC, London (GB)

(21) Appl. No.: 13/202,974

(22) PCT Filed: Feb. 19, 2010
(86) PCT No.: PCT/GB2010/000293

§ 371 Date: Oct. 5, 2011

(30) Foreign Application Priority Data
Feb. 24, 2009 (GB) 0903032.1

(51) Int. Cl. A61M 1/00; B26D 3/12

(52) U.S. Cl. 604/319; 83/13

(57) ABSTRACT

Embodiments disclosed herein relate to a wound dressing, suitable for use in negative pressure wound therapy, comprising a body of porous material, the body of porous material comprising a plurality of cuts which provide regions of flexibility within the body. Further embodiments relate to methods of manufacturing and method of use of such wound dressings.

The present invention relates to improved wound dressing materials. In particular the invention relates to wound dressing materials with improved drapeability, which allows them to more easily conform to the shape of a surface over which they are placed. Especially, but not exclusively, the present invention relates to foam materials, especially those suitable for use in negative pressure wound therapy (NPWT).

NPWT is a relatively new treatment for open wounds. Typically in NPWT the wound cavity or surface is filled or covered with a material that allows the transmission of a partial vacuum (i.e. does not completely collapse) to the wound bed when a negative pressure is applied to the wound area, and also allows fluids to pass from the wound bed towards the source of negative pressure. There are two primary approaches to NPWT, gauze or foam types. The gauze type (also referred to as the Chariker-Jeter technique) involves the use of a drain wrapped in gauze topped by a sealed dressing. The foam type involves the use of foam placed over or in the wound. The present invention is directed primarily, but not exclusively, towards the foam type of NPWT.

In foam based NPWT the wound is filled or covered with a porous, compressible foam packing material and covered over and sealed with flexible sheet (a drape) that is fairly impermeable to fluids. A tube is inserted under or through the drape into the wound site and its distal end is connected to a vacuum source (commonly a pump). The wound cavity, enclosed by the drape and tissue, contracts under the force of atmospheric pressure and compresses the packing material visibly. Gross tissue movement ceases after a few tens of seconds and fluid flow from the wound (withdrawn from the tissue) ensues. The fluid is transmitted through the packing material and up the vacuum tube to a collection receptacle positioned between the distal end of the tube and the vacuum source. The material mechanically supports the tissue to which it is applied, and also allows the free flow of fluids away from the site when a vacuum is applied, even when compressed. A good material for this application is hydrophobic, reticulated polyurethane foam of very high free internal volume. However, articles of high free internal volume tend to be poorly drapeable due to the requirement for their structure to mechanically support their high free internal volume, and this is the case in current foams applied in NPWT.

The foams, which in other regards are near optimal for NPWT, are very poorly conformable to the site of application especially prior to the application of a covering drape and vacuum being applied. This is true for cavity wounds of concave geometry, and surface wounds, e.g. to the extremities, of convex geometry.

Monolithic articles of high free internal volume are able to support their own external dimensions by virtue of their mechanical properties, i.e. they are relatively stiff. This factor has the side effect of making such solid objects poorly drapeable and this is not desirable for all applications. For some applications, the mechanical integrity of the structure is not required at the scale of the monolith itself.

For medical applications involving the application of articles to the body, both good drapeability and good resistance to compression under loading are desirable attributes.

According to the present invention there is provided a wound dressing comprising a body of porous material, the body of porous material comprising a plurality of cuts which provide regions of flexibility within the body.

While the plurality of cuts provide regions of flexibility within the body, they are not capable of rendering portions of the body frangible, such that the portions are relatively easily severable from the body.

Preferably the porous material is a wound packing foam suitable for use in negative pressure wound therapy (NPWT). Particularly suitable foams for NPWT include polyurethane foam, typically reticulated polyurethane foam of very high free internal volume, e.g. 80% or higher, preferably 90% or higher free internal volume. Typical foams used in NPWT have porosities in the range 30-60 ppi (pores per inch) and mean pore diameters in the range 300-800 μm. However, other suitable foams are known in the art and may be equally employed. In general suitable foams have an open porous structure, to allow transmission of the negative pressure to the wound bed, and sufficient mechanical strength to prevent the negative pressure (typically approximately 80-125 mm Hg below ambient atmospheric pressure) from collapsing the structure of the foam.

Suitably the cuts are slits. The term slit is intended to mean a cut which is generally long and thin, and preferably straight and linear. In practice, slits in foam are typically effectively 2-dimensional as the resilience of the foam means that the slit is essentially closed unless the material is stretched. Suitably the slits are from 10 mm to 70 mm in length, preferably from 20 mm to 50 mm, especially from 25 mm to 40 mm. Slits of around 30 mm have been found to be particularly effective in a typical NPWT, though there is of course scope to vary this.

Providing cuts, e.g. slits, confers macroscopic flexibility, while not substantially affecting the microscopic mechanical properties of the body, i.e. to resist compression under negative pressure. This flexibility allows the body of porous material to drape more easily, i.e. to conform to the shape of the wound to be dressed.

In a preferred embodiment, the body comprises at least one linear series of slits, each slit being separated from an adjacent slit by a gap. By “linear series of slits” it is intended to mean a plurality of generally linear slits, each slit generally aligned along a straight line in a series, with a gap separating each slit from adjacent slits in the series. Generally it is preferred that the gaps are regularly sized.

Preferably the body comprises two or more parallel linear series of slits, each linear series being spaced from the adjacent linear series. Preferably the spacing between the adjacent linear series is regular.

It will be clear to the person skilled in the art that the size of the gap between the slits, and the spacing between adjacent series, must be sufficient such that the material retains structural integrity sufficient for it to be handled, used in therapy and removed thereafter without breaking up. If the gaps and spacing were too small, the body would be too weak to achieve this. On the other hand, there is a desire to maximise the size and density of the slits to maximise drapeability. It is preferred that the minimum size of the gaps and/or spacing should certainly be no less than the average pore diameter. It is more preferred that gaps and/or spacing are at least 5 times the average pore diameter; given that the average pore diameter for NPWT foam is in the range of 300-800 μm, this gives a gap or spacing of 1500 to 4000 μm, i.e. 1.5 to 4 mm. A gap of around 3 mm has been found to be particularly effective in a typical NPWT, though there is of course scope to vary this.

It is preferable that the spacing between adjacent linear series of slits is kept reasonably small to provide the desired amount of flexibility and hence drapeability. Accordingly, it is preferred that the spacing is not more than 50 times the average pore diameter (typically from 15 mm to 40 mm depending on pore density), preferably not more than 30 times than average pore diameter (typically from 9 mm to 24 mm depending on pore density). A spacing of around 3 mm has been found to be particularly effective in a typical NPWT, though there is of course scope to vary this.

It is preferred that, where two or more parallel linear series of slits are provided, adjacent series are linearly offset relative to each other, i.e. it is preferred that the slits and gaps in adjacent series do not line up, but are staggered. To put it another way, adjacent series may be out of phase with each other. In one embodiment adjacent series are offset such that the centre point of a slit in one series is aligned approximately with the gap in an adjacent series—similar to the way layers of bricks are offset in a wall. There is of course scope to vary the amount of offset.

This offset of adjacent series of slits allows for particularly good drapeability. When the body is curved the slits open up to form a lattice type structure which is particularly suited to provide good flexibility, and hence drapeability.

The slits may suitably pass completely through the thickness of the body. Alternatively the slits may pass only partially though the thickness of the body, provided they pass through far enough to provide the desired flexibility to the body. In general it is preferred that the slits pass at least half way through the thickness of the body, preferably at least three quarters of the way through the body, and especially substantially all the way through the body.

The present application is particularly suited to a wound dressing material comprising a body of porous material which is relatively thin, although it could be used to impart flexibility to a body which is thick. In particular, the present invention is particularly suited to a sheets of porous material which have a thickness of from 5 mm to 75 mm, preferably from 10 mm to 50 mm, especially from 15 to 40 mm, most preferably from 20 to 35 mm. In a very thin sheet (less than 5 mm) drapeability of the sheet is not such an issue, and where the thickness is greater than about 75 mm, providing a plurality of cuts becomes less effective as a means of introducing flexibility. Thus the ranges set out above represent sheets of thicknesses which are particularly well suited to the present invention. The other dimensions of the sheet are not particularly significant, although it may be observed that NPWT foam is typically sold in generally cuboid sheets with the dimensions of the edges of largest face being between 100 and 200 mm, e.g. a sheet measuring 100×200×30 mm is fairly typical.

It is generally preferred that the cuts are provided passing through the shortest dimension of the body. In particular it is preferred that the cuts (e.g. slits) are provided passing between the two largest faces of a cuboid body (e.g. sheet), and especially that the cuts are perpendicular to the largest faces.

Where two or more parallel linear series of slits are provided, as set out above, this provides for good flexibility in a single direction of curve. When a body is curved around a surface, the inside of the body is subject to compression and the outside of the body is subjected to tension (inside and outside being defined relative to the curve). In wound dressings according to the present invention the cuts allow the body to stretch when under tension, the cuts allowing deformation of the shape of the body (e.g. into a lattice), and this allows the body to easily adapt to the desired curve. Where the cuts comprise slits in a single orientation, the body will be well adapted to curving in a direction which causes tension perpendicular to the slits, but will be less well adapted to curving in another direction, i.e. where the tension produced is parallel to the slits. Therefore, the body is well suited to drape around a body with a single curvature (e.g. generally cylindrical), but not so well suited to draping around a more complex curved object such as a generally spherical shape.

In a further embodiment of the present invention, the body comprises a second set of slits passing at least partially through the body in a different orientation (preferably perpendicular) to the slits in the at least one linear series of slits described above (the “first set of slits”). It is preferred that the second set of slits also pass between, and are perpendicular to, the largest faces of the body. Additional slits provided in this manner allow the body to curve more easily in a second direction thus making it more suited to adapting to a complex curved surface, i.e. curved in two planes.

In a preferred embodiment of the invention at least some of the slits in both orientations intersect. Suitably the intersection of the slits effectively provides a two-dimensional (2-D) slit. Preferably the 2-D slit has an H shape, with a two parallel slits in one orientation being intersected at their mid-points by a third slit in a perpendicular orientation. Such an H-shaped 2-D slit is well suited to the provision of a relatively dense array of 2-D slits in the body. For example, rows of H-shaped slits, each adjacent slit within the row alternating in orientation by 90 degrees, can be packed closely together, while obeying the minimum spacing requirements set out above.

In a preferred embodiment of the invention, the body comprises a first set of parallel linear series of H-shaped slits in a first orientation and a second set of parallel linear series of H-shaped slits in a second orientation. Preferably the second orientation is perpendicular to the first orientation. Preferably adjacent series within a set are offset relative to one another. Such an arrangement allows very close packing of the H-shaped slits, whilst maintaining a suitable spacing between neighbouring slits to retain the overall structural integrity of the body. To allow close packing of the slits it is preferred that the length of the two parallel slits (the “sides”) of the H-shaped slit are shorter than the perpendicular slit (the “cross-piece”) of the H-shaped slit. For example, the “cross-piece” may be from 10 mm to 70 mm in length, preferably from 20 mm to 50 mm, especially from 25 mm to 40 mm, most preferably 30 mm and the “sides” are preferably around 6 mm shorter than the “cross-piece”, especially around half the length of the “cross-piece”. Where the “sides” of the H are shorter than the “cross-piece”, it allows the “sides” of H-shaped slits in one orientation to nest within the area bounded by “sides” and “cross-piece” of the H-shaped slit in the other orientation. This allows for very close packing of the H-shaped slits.

It should be noted that while a body having an array of H-shaped 2-D slits is a preferred embodiment of the present invention, it is possible to achieve a suitable 2-D array using other shapes, such as cruciform slits, or the like.

It should be noted that the cuts, e.g. slits, of the present invention can be provided by cutting a preformed body of foam, or the cuts could be provided during the process in which the body is manufactured (e.g. moulding). It is preferred that the cuts are provided by die cutting a preformed body.

In a further aspect, the present invention provides a method of manufacturing a wound dressing, the method comprising the steps of;
providing a body of a porous material; and
providing a plurality of cuts which provide regions of flexibility within the body.

It is preferred that the cuts are provided by die cutting using an array of suitably sized and shaped blades to provide the desired cuts.

Details of preferred cuts, e.g. slits, are set out above, and it will be obvious to the person skilled in the art how to provide a suitable blade to achieve the desired cut.

The cuts may be provided all the way through the body, or they may only pass partially through the body.

The cuts may be formed in a batch, or they may be formed in a flow process.

In a further aspect the present invention provides a method of dressing a wound comprising administering a wound dressing as set out above to the wound.

Preferably the method provides the step of applying a negative pressure to the wound through the wound dressing material, i.e. the method is NPWT. In general this can be achieved by providing a substantially fluid impermeable sheet over the wound and wound dressing, thus defining a sealed volume, and applying a negative pressure inside said sealed volume. The seal need not be completely hermetic, but should be sufficient to allow a suitable negative pressure to be sustained. The source of negative pressure, e.g. a pipe form a vacuum pump, can be provided at a position such that it draws fluids from the wound bed through the wound dressing material.

Suitably the negative pressure is in the range of from 80 to 125 mm Hg below ambient atmospheric pressure.

In a further aspect the present invention provides the use of a wound dressing material as set out above in wound treatment, especially NPWT.

Embodiments of the present invention will now be described, by way of example only, with reference to the accompanying drawings, in which:

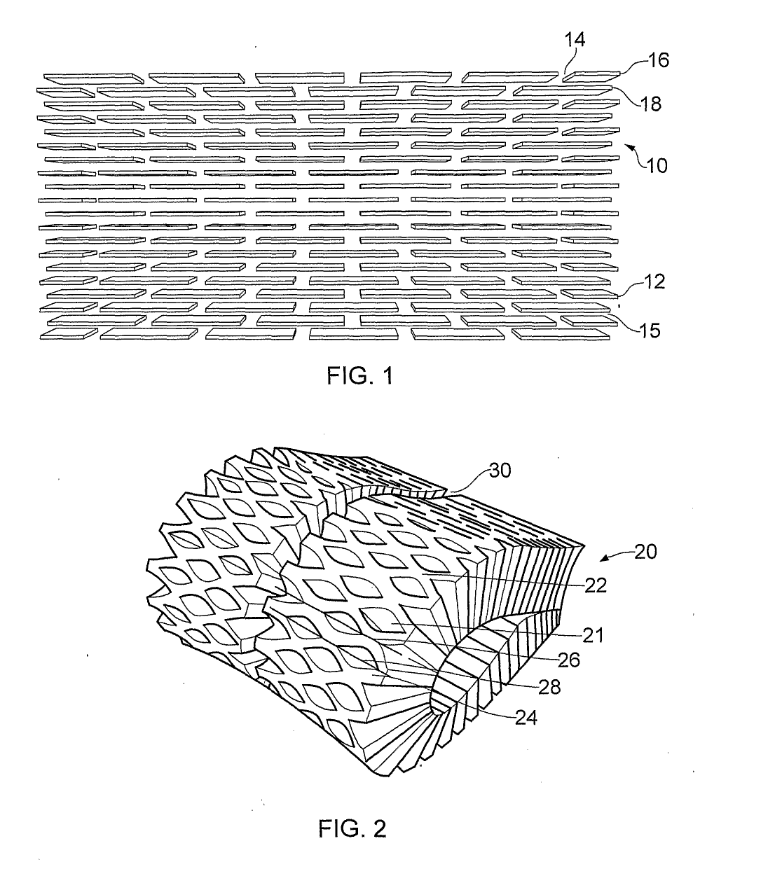

FIG. 1 shows an array of blades adapted to form slits in a body of a wound dressing material according to the present invention;

FIG. 2 shows a body according to the present invention curved in a first direction;

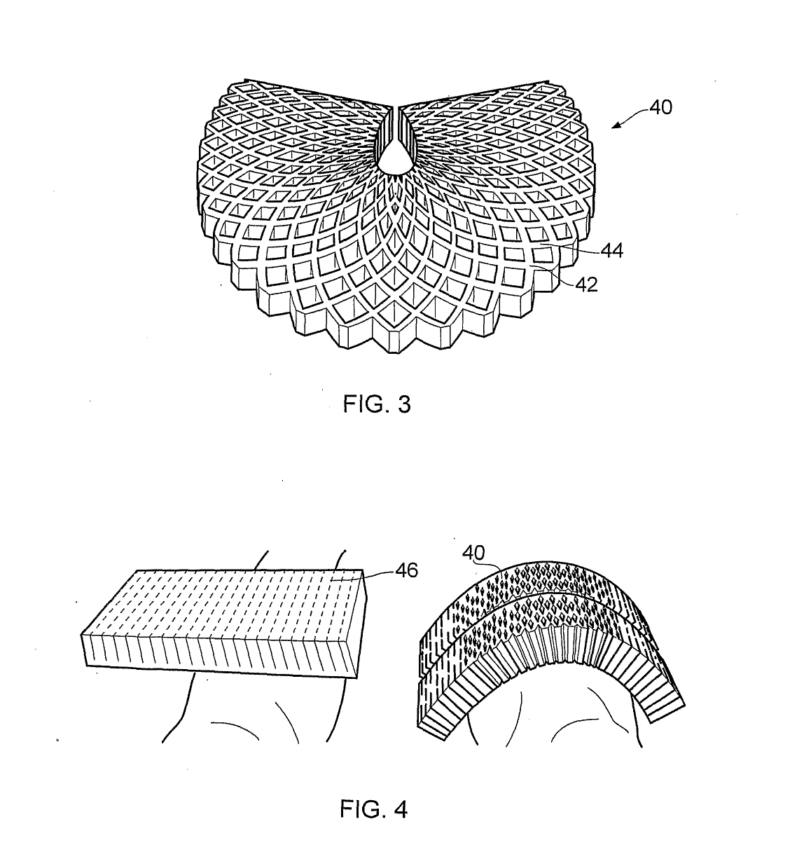

FIG. 3 shows a body according to the present invention curved in a second direction;

FIG. 4 shows a comparison of a body of foam according to the present invention with an un-cut body of foam;

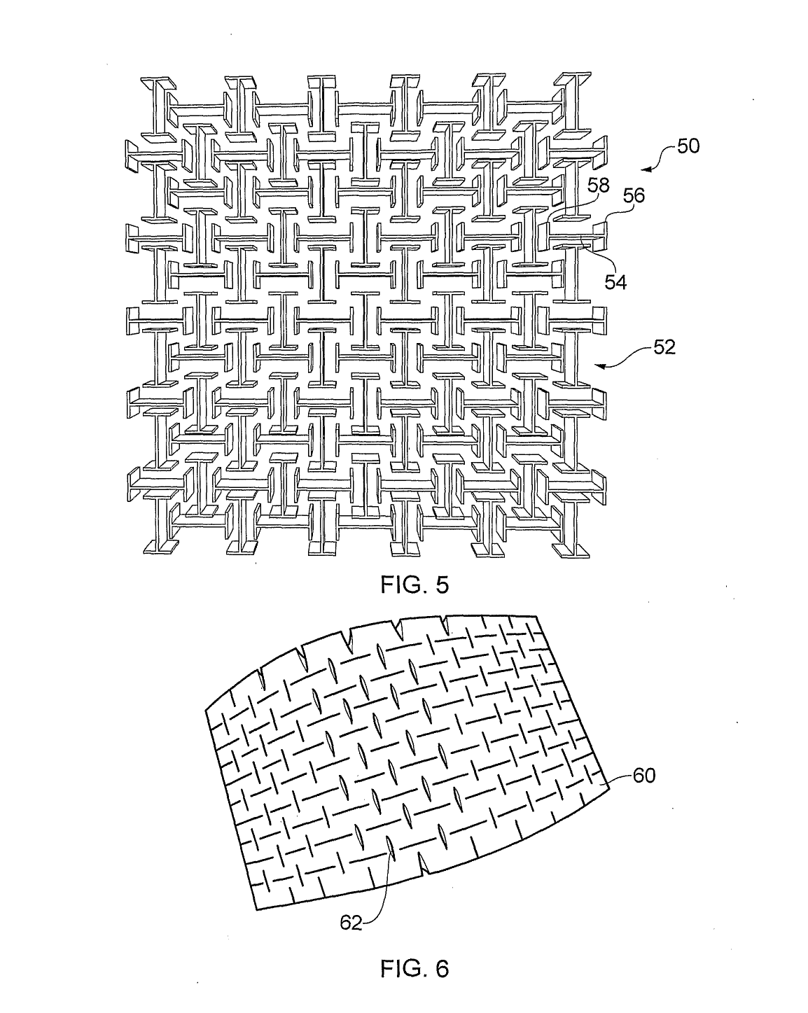

FIG. 5 shows a second array of blades adapted to form cuts in a body of a wound dressing material according to the present invention;

FIG. 6 shows a body according to the present invention cut with the array of blades of FIG. 5; and

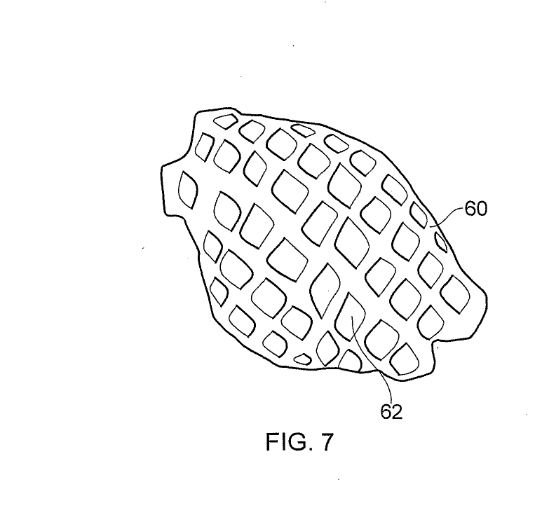

FIG. 7 shows the body of FIG. 6 curved in two dimensions.

An array of blades (10) mounted on a board is shown in FIG. 1. Each blade (12) is a straight thin blade 30 mm long, and having a depth of approximately 30 mm. The blades are arranged in 20 parallel linear series of blades (16,18), each series comprising a row of blades (12) arranged longitudinally, with a gap (14) of 3 mm between each blade (12) in the series. Each series is spaced from the adjacent series by a 3 mm spacing (15). Furthermore, adjacent series (16,18) are staggered relative to one another such that the gap between the blades on one series (16) aligns with the midpoint in the adjacent series (18). Accordingly, the blades within the array (10) are arranged like the bricks in a wall. Given this offset arrangement, it is convenient that at the end of a series where a full 30 mm blade would extend beyond the dimension to be cut, blades of 15 mm length are provided; this allows for a neater array—once more, this is akin to half bricks at the end of a row in a wall. Full length blades could be used at the ends, provided they would not be problematic in the cutting process.

A body of NPWT foam (20) measuring 200×125×30 mm is cut using the array (10). It is cut by driving the array of blades (10) through the body (20) in a die cutting operation. This can be achieved using a press, typically a hydraulic press (not shown), also known as a clicker press. The blades are driven perpendicularly into and through the largest face of the body (20), and perpendicular thereto, to form a plurality of slits therein. The slits (21) formed are arranged in a plurality of parallel linear series (26,28) of slits, each comprising slits (21) 30 mm long separated by gaps (22), where material is left un-cut, which are 3 mm long. Each series is separated by a spacing (24) 3 mm in width. When the body (20) is curved, as shown in FIG. 2, the slits (21) open up to form a lattice structure. Tension in the outer region of the body (20) as a result of the curving process is relieved through deformation of the body (20) which is facilitated by the slits (21) provided therein. The arrangement of parallel offset linear series of linear slits is particularly suited to this as it form a regular lattice structure, as shown in FIG. 2.

In the embodiment shown in FIG. 2, an additional partial cut (30) has been made running the length of the middle of the largest face of the body (20), perpendicular to the slits. This allows the body (20) to be easily split in two if this is desirable.

FIG. 3 shows another body (40) cut using the array of blades of FIG. 1, this time without the additional cut (30). The body has been curved in a different manner to that in FIG. 2. In this case the body has been bent back on itself along its longest side, i.e. the 200×300 mm face has been curved back on itself. The body (40) has opened via the slits (42) into an open lattice structure. This type of curving of the body (40) is not generally useful for a wound dressing application, but does serve to demonstrate the flexibility and strength of the body (40).

FIG. 4 further demonstrates the ability of a body according to the present invention (40) to drape over a surface, in this case a leg, when compared to an uncut body (46).

FIG. 5 shows an array (50) of blades adapted to form cuts in a body of foam in two orientations, the orientations being perpendicular to each other. As with the array (10) in FIG. 1, the blades have a depth of 30 mm. However, in the array (50) comprises H-shaped blades (52) comprising a first blade element 30 mm long (54) (also termed “cross-piece”), with second (56) and third (58) blade elements (also termed “sides”) 15 mm long located at the end of the first blade element (54), each end of the first blade element intersecting with the midpoint of the second and third blade elements, thus defining a “wide H-shaped” blade. The array is made up of first set of eleven parallel linear series of H-shaped blades in a first orientation (called X for convenience) and a second set of eleven parallel linear series of H-shaped blades in a second, perpendicular orientation (called Y for convenience). Adjacent series within each set are offset in exactly the same manner as for linear blades. As can be seen from FIG. 5, the blades are spaced and arranged such that a close packing of the blades as achieved, but each blade is always approximately 5 mm or so from the nearest neighbouring blade. It can be seen that the “side” of a blade in the X-orientation nests within the region defined by the “cross-piece” and “sides” of a blade in the Y-orientation.

Such an array is suited to forming slits in a body to allow draping in two planes.

FIG. 6 shows a body (60) formed by cutting with the array of FIG. 5. The slits (62) are formed by pressing the array of blades (50) through the body (60) in the same manner as described above. H-shaped slits (62) are formed in the body (60) corresponding to the array of blades (50). As shown in FIG. 7, the body (60) is well adapted to curving in complex shapes.

It should be noted that the present description has focussed on bodies formed by a batch die cutting process. There are of course numerous ways of forming cuts in a body of porous material (e.g. laser cutting, high pressure liquid cutting), or the cuts could formed when the body itself is formed (e.g. during a moulding process). Furthermore, these methods could be applied in a flow process rather than a batch; this might be more efficient for large production runs. All such variations are within the scope of the present invention.

Furthermore, it should be noted that, while the exemplified embodiments form particularly preferred embodiments with excellent drapeability, it is quite possible that other arrangements of cuts will provide satisfactory results.

1. A wound dressing comprising a body of porous material, the body of porous material comprising a plurality of cuts which provide regions of flexibility within the body, wherein the porous material is a wound packing foam suitable for use in negative pressure wound therapy, the foam having a free internal volume of 80% or higher.

2. (canceled)

3. The wound dressing of claim 1 wherein the porous material is a wound packing foam suitable for use in negative pressure wound therapy having a porosity in the range 30-60 pores per inch and mean pore diameter in the range 300-800 μm.

4. The wound dressing of claim 1 wherein the cuts are slits.

5. The wound dressing of claim 4 wherein the cuts are slits from 10 mm to 70 mm in length.

6. The wound dressing of claim 4 wherein the body comprises at least one linear series of slits, each slit being separated from an adjacent slit by a gap.

7. The wound dressing of claim 6 wherein the body comprises two or more parallel linear series of slits, each linear series being spaced from the adjacent linear series.

8. The wound dressing of claim 6 wherein the minimum size of the gaps is at least as big as a mean pore diameter of the wound packing foam.

9. The wound dressing of claim 8 wherein the size of the gaps is at least 5 times the mean pore diameter.

10. The wound dressing of claim 8 wherein the spacing between adjacent linear series of slits is not more than 50 times the mean pore diameter.

11. The wound dressing of claim 4 comprising two or more parallel linear series of slits, wherein that adjacent series are linearly offset relative to each other.

12. The wound dressing of claim 1 wherein the cuts pass at least half way through the thickness of the body.

13. The wound dressing of claim 1 wherein the body comprises a sheet of porous material having a thickness of from 5 mm to 75 mm.

14. The wound dressing of claim 6 comprising a second set of slits passing at least partially through the body in a different orientation to the at least one linear series of slits.

15. The wound dressing of claim 14 wherein at least some of the slits in both orientations intersect to provide a two-dimensional (2-D) slit.

16. The wound dressing of claim 15 wherein the 2-D slit has an H shape, with a two parallel slits in one orientation being intersected at their mid-points by a third slit in a perpendicular orientation.

17. The wound dressing of claim 16 wherein the body comprises a first set of parallel linear series of H-shaped slits in a.first orientation and a second set of parallel linear series of H-shaped slits in a second orientation.

18. The wound dressing of claim 17 wherein the second orientation is perpendicular to the first orientation.

19. The wound dressing of claim 17 wherein adjacent series within a set are offset relative to one another.

20. The wound dressing of claim 17 wherein the two parallel slits of the H-shaped slit are shorter than the perpendicular slit, thus allowing the two parallel slits of H-shaped slit in one orientation to nest within the area bounded by two parallel slits and perpendicular slit of the H-shaped slit in the other orientation.

21. A method of manufacturing a wound dressing, the method comprising the steps of
providing a body of a porous material, wherein the porous material comprises a wound packing foam suitable for use in negative pressure wound therapy; and
making a plurality of cuts which provide regions of flexibility within the body of porous material.

22. The method of claim 21 in which the cuts are provided by die cutting using an array of suitably sized and shaped blades to provide the desired cuts.

23. A method of dressing a wound comprising:
placing a body of porous material over or into a wound, the body of porous material comprising a wound packing foam having a free internal volume of 80% or higher, the body of porous material further comprising a plurality of cuts to provide regions of flexibility within the body of porous material;
placing the porous material over the wound so as to substantially conform to the contours of the wound;
sealing the wound with a flexible, substantially fluid-impermeable drape; and
applying negative pressure to the wound through a tube connected through or under the drape.

24. (canceled)

25. The method of claim 23 wherein the negative pressure is applied in the range of from 80 to 125 mm Hg below ambient atmospheric pressure.

26. (canceled)

27. The method of claim 23, wherein the porous material has a porosity in the range 30-60 pores per inch and a mean pore diameter in the range 300-800 μm.

28. The wound dressing of claim 1, wherein the foam has a free internal volume of 90% or higher.

29. The method of claim 21, wherein the plurality of cuts form a first linear series of slits, each slit being separated by a gap.

30. The method of claim 27, further comprising making at least one additional plurality of cuts in a linear series of slits, wherein the at least one additional linear series of slits is spaced apart from the first linear series of slits.

31. The method of claim 21, wherein the plurality of cuts form H-shaped slits, each H-shaped slit comprising two parallel slits in one orientation being intersected at their mid-points by a third slit in a perpendicular orientation.

32. A wound treatment system, comprising:
the wound dressing of claim 1;
a flexible, substantially fluid-impermeable sheet configured to be provided over the wound dressing; and
a source of negative pressure configured to draw fluids from a wound bed through the wound dressing.
